(19) United States
(12) Patent Application Publication
(10) Pub. No.: US 2024/0248254 A1
(43) Pub. Date: Jul. 25, 2024

(54) DEVICE FOR AMPLIFYING LIGHT BY MEANS OF REFLECTIONS IN CONDUITS

(71) Applicant: Johann-Marius MILOSIU, Erlangen (DE)

(72) Inventor: Johann-Marius MILOSIU, Erlangen (DE)

(21) Appl. No.: 18/575,340

(22) PCT Filed: Jun. 21, 2022
(86) PCT No.: PCT/DE2022/000068

§ 371 Date: Dec. 29, 2023

(30) Foreign Application Priority Data
Jun. 30, 2021 (DE) 10 2021 003 356.6

(51) Int. Cl. G02B 6/10; G02B 5/08; G02B 5/124; G02B 17/00

(52) U.S. Cl. CPC G02B 6/102; G02B 5/0891; G02B 5/124; G02B 17/002; G02B 17/004

(57) ABSTRACT

The present invention utilizes a system of primarily metallic pairs of mirrors, at 90° angles to each other and at 45° angles to the conduit axis, which cover only one inner wall of a fluid conduit in a parallel arrangement, while the other walls are simply mirrored. The useful radiation from e.g. UV light-emitting diodes comes through small breakthroughs in the valley bottom between two mirrors of a pair concentrated into the conduit. The double reflection in the pairs of mirrors sends the radiation back to where it came from in each section of the conduit—at least the axial component—while at the same time a lateral offset of the beams causes a chain reflection around the walls of the conduit, although this only in a defined area takes place, which has approximately the same extent as the beam cone of the light source on the opposite wall, which is also slightly concave in order to achieve better bundling of the rays. The radiation level in the delimited section intensifies even more chain reflections can take place.

This makes it possible to achieve considerably high irradiation values in the conduit even with a relatively weak UV light source and thus ensure very effective disinfection of the fluids contained therein.

### TECHNICAL FIELD OF THE INVENTION

The invention refers to a device for light amplification by means of multiple reflections in conduits.

### STATE OF THE ART

Devices that serve a similar purpose are quite well known.

There are channels or conduits for fluids that have mirrored walls.

They serve to allow the radiation from light sources to work longer in the conduits and the medium they contain through reflection. That's how it is, for example. B. DE 202021 100238 U1 or EP 3 378 501 A1. The disadvantage of this type of mirroring is that after a few reflections the radiation is redirected in the axial direction and the desired effects, such as e.g. the intensification of radiation when disinfecting the air from the pipes is reduced by UV radiation and a greater power and number of UV light sources is required. From DE 17 64 878 A and DE 12 04 747 A, devices used in the field of laser technology and methods for light amplification are known, in which light is reflected several times from walls, and in circuits within a solid-state line over and over again the same path within a defined area. These devices can only amplify intrinsic and strictly parallel or coherent rays and are not suitable as amplifiers for diverging light and especially UV emitters.

### SUMMARY OF THE INVENTION

In one exemplary embodiment, the present invention comprises a special arrangement of parallel mirror pairs which are at 90° angles to one another, with each mirror at a 45° angle to the axis and are attached to only one of the four conduit walls, while the other walls are simply mirrored to be used.

These pairs of mirrors are oriented transversely to the longitudinal axis of the respective conduit. This system of mirrors also enables the radiation of small-area and relatively strongly diverging light emitters, such e.g. UV light-emitting diodes to reflect so that the rays remain in a limited area of the conduit. The double reflection in the pairs of mirrors, which are at 90° angles to each other, always sends the axially directed portion of the radiation back to where the radiation comes from; the lateral portion of the radiation only leads to a lateral offset in the channel. In this way it is ensured that the radiation only occurs in a narrowly limited area of the conduit remains, where it reaches bigger intensity, depending of how much the reflectance of the mirror is. If you fill these channels with a fluid that is transparent to radiation, such for example air for UV radiation, a relatively weak light source can cause the radiation density in the channel to reach such high values that it is completely sufficient for the desired purpose—usually disinfection —.

In most cases, a rectangular cross-section of the conduits is chosen, but others, such as polygonal or round profiles, can also be used. To allow unhindered reflection of radiation, metallic mirrors without a cover or protective layer are required. Since the effect of double reflection independent of the size of the mirror is, very small and elongated mirror segments can be used, which leads to easy production of such mirror coatings on conduits. The usually disturbing effect of divergence when emitting radiation is insignificant with this type of multiple reflection. Since the radiation density when using this device, depending on the degree of reflection of the mirror coating, reaches significantly high values that are many times greater than the radiation intensity of the mere light source, it can be referred to as a Light Amplifier using Reflections in the Local Area, or LARLA for short.

### BRIEF DESCRIPTION OF DRAWINGS

An exemplary embodiment of the device according to the invention is shown in the drawings and is explained in more detail below.

It shows:

FIG. 1 Longitudinal section through the device

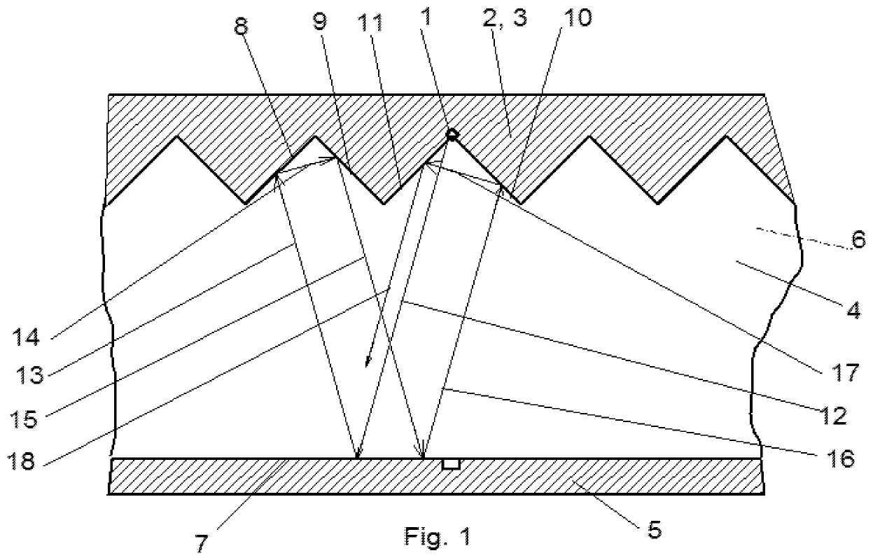

FIG. 2 Cross section through the device

### DETAILED DESCRIPTION OF SOME EMBODIMENTS OF THE INVENTION

The present invention relates to a device for light amplification by means of reflection in conduits with an approximately rectangular cross-section, the walls being mirrored and provided with regularly arranged light sources 1 in such a way that the light rays are reflected several times by the walls.

The light from a quasi-point light source 1 is amplified by multiple special reflections in a limited area of a conduit 3 for the transport of fluids that are transparent to the light used, by a special design or geometry of the mirrored conduit walls 2, 4, 5, 6

is used, which forces the light rays to mostly always take the same path and cause repeated illumination of the same room or surfaces, so that the radiation along the conduit 3 is prevented from disappear.

The reflection properties of the walls are chosen differently in such a way that only one wall tends to send the rays in the direction from which they come, while the other walls reflect the rays more or less normally, with the effect that no rays reach a defined area of the conduit 3 being able to leave.

The radiation density, depending on the degree of reflection of the mirror coating, reaches significantly high values, which exceeds the radiation intensity of the mere light source many times over and the device can therefore be referred to as a light amplifier by reflections in the local area, or LARLA for short.

Any beam 12, located in an axial plane which is also perpendicular to the walls 2, 5, from a light source 1 present in the wall 2 of the conduit 3 falls on a mirror surface 7 of the wall 5 and is reflected as a beam 13 and falls on one mirror surface 8, which is part of a double mirror pair 8, 9, from where it falls as a beam 14 onto the other half of the mirror pair 9, from where it goes back to the wall 5 as a beam 15 parallel to the beam 13.

The beam 15 from the mirror surface 7 of the wall 5 goes as a beam 16 parallel to the first beam 12 back to the wall 2, in order to go from the double mirror surface 10,11 back to the wall 5 as a beam 17 and then as a beam 18, the beam 18 not only being parallel to the first beam 12, but also being very close to it, so that from this point on the play of reflections occurs as already described repeated.

It can be seen that the rays cannot leave the delimited area.

Other rays from the light source 1 that are not in a normal plane to the walls 2, 5, with additional lateral reflections on the walls 4, 6, ultimately experience a similarly limited propagation in the space of the conduit 3 as the previously analyzed rays.

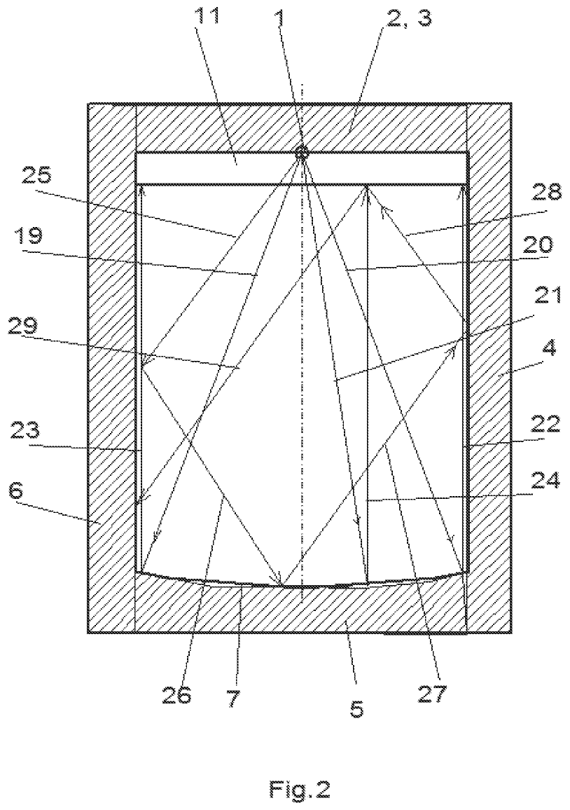

In a plane transverse to the conduit 3, as can be seen in FIG. 2, the mirror-coating 7 of wall 5 force a concentration of the divergent rays 19, 20, 21, which go parallel to the walls 4, 6 to the mirror-coating of the wall and from where they will be reflected back to the wall 5 and from where they bundled to the light source 1 go, from where the beam reflections take place again as already described.

The rays outside the cone marked by the rays 19, 20 experience additional reflections on the walls 4, 6, but remain in the delimited area.

An inner wall of a quasi-rectangular conduit 3 is covered with a row of mirror pairs 8, 9, . . . arranged in parallel, these mirror pairs consisting of individual elongated mirrors at a 90° angle to one another, which are also at a 45° angle to the conduit axis.

A small-area light source 1 is placed in the valley between the mirror elements of a suitable pair of mirrors, or if it is placed outside the mirror plane, its radiation is sent into the conduit through a breakthrough of small spatial extent.

The side surfaces 4, 6 of the conduit 3 are mostly smooth and mirrored, while the wall 5, which is opposite the wall 2 with the double mirror pairs, has a cylindricity along the longitudinal axis of the conduit 3, with a radius about twice as large as that distance between walls 2 and 5.

In another exemplary embodiment of the invention, a polygonal or even round cross section of the conduit 3 is used instead of a rectangular one, with the property of the limited axial spread of the beams being approximately retained.

All pairs of mirrors used are usually metallic mirrors without a cover or protective layer.

Relatively very small and elongated mirror segments are primarily used, which leads to easy production of such mirror coatings on the conduit 3.

What is claimed is:

1. A device for amplifying light by means of reflection in conduits with an approximately rectangular cross-section, walls being mirrored and provided with regularly arranged light sources (1), such that light rays are reflected several times by the walls, characterized in that,
a. an amplification of the light from a quasi-point light source (1) by multiple special reflections in a limited area of a conduit (3) for the transport of fluids, that are transparent to the light used, takes place by a special design or geometry of mirrored conduit walls (2, 4, 5, 6), which force the light rays to always take the same path and cause repeated illumination of the same room or area, so that the radiation is prevented from disappearing along the conduit (3),
b. reflection properties of the walls are chosen differently in such a way, that only one wall tends to send the rays in a direction from which they come, while other walls reflect the rays more or less normally, with the effect that no rays of a defined area of the conduit (3) can leave it; and
c. whereby a radiation density, depending on a degree of reflection of a mirror coating, reaches significantly high values, which exceeds the radiation intensity of the light source many times over.

2. The device according to claim 1, characterized in that,
a. a first beam (12), located in an axial plane, which is also perpendicular to the walls (2, 5) of the light source (1) present in the wall (2) of the conduit (3) on a mirror surface (7) of the wall (5) falls and is reflected as a beam (13) and falls on a mirror surface (8), which is part of a double mirror pair (8, 9), from where it falls as a beam (14) onto the other half of the double mirror pair (9) falls, in order to go from there as a beam (15) parallel to the beam (13) back to the wall (5,6);
b. wherein the beam (15) goes from the mirror surface (7) of the wall (5) as a beam (16) parallel to the first beam (12) back to the wall (2) in order to be reflected from a double mirror surface (10, 11) as beam (17) and then as a beam (18) to be sent to the wall (5), whereby the beam (18) is not only being parallel to the first beam (12), but also being very close to it, so that from this moment reflections repeats itself, whereby the rays cannot leave a demarcated area;
c. whereby other rays from the light source (1), which are not in a normal plane to the walls (2, 5,) ultimately experience a similarly limited propagation in the space of the conduit (3) with additional lateral reflections on the walls (4, 6), like rays (15) to (18);
d. wherein in a plane transverse to the conduit (3), the mirror surface (7) of the wall (5) causes a concentration of diverging rays (19, 20, 21), so that they go quasi-parallel to the walls (4, 6) to mirroring the wall (2), in order to go from there again to the wall (5), from where they go somewhat bundled towards the light source (1), from where the path of the rays takes place again; and
e. wherein the rays outside a cone, marked by rays (19, 20), experience additional reflections on the walls (4, 6), but remain in the demarcated area.

3. The device according to claim 1, characterized in that,
a. an inner wall side of the conduit (3) is covered with a double mirror pair (8, 9) arranged in parallel, these double mirror pairs consisting of individual elongated mirrors at 90° angles to one another, which are also at a 45° angle to a conduit axis;
b. wherein a small-area of the light source (1), which is a part of a series of such regularly installed light sources, in a valley which is placed between mirror elements of a suitable pair of mirrors, or if it is placed outside a mirror plane, sends its radiation into the conduit (3) through a breakthrough of small spatial extent; and
c. wherein side surfaces of the walls (4, 6) of the conduit (3) are smooth and mirrored, while the wall (5), which is opposite the wall (2) with the double mirror pairs, has a cylindricity to a longitudinal axis of the conduit (3), wherein the cylindricity has a radius approximately twice as large as a distance between the wall (2) and (5).

4. The device according to claim 3, characterized in that, an approximately rectangular and polygonal or even partially rounded cross section of the conduit (3) is used, the property of a limited axial spread of the beams being approximately retained.

5. The device according to claim 2, characterized in that,
a. all pairs of mirrors used are metallic mirrors without a cover or protective layer,
b. wherein relatively very small and elongated mirror segments are primarily used, which leads to easy production of such mirror coatings on the conduit (3).

6. The device according to claim 1, characterized in that, for use the conduit (3) is filled with air in order to achieve effective disinfection with the aid of one or a few weak UV light sources.

7. The device according to claim 2, characterized in that,
a. an inner wall side of the conduit (3) is covered with the double mirror pair (8, 9) arranged in parallel, these double mirror pairs consisting of individual elongated mirrors at 90° angles to one another, which are also at a 45° angle to the conduit axis;
b. wherein the light source (1), which is the part of a series of such regularly installed light sources, in the valley which is placed between the mirror elements of suitable pair of mirrors, or if it is placed outside the mirror plane, sends its radiation into the conduit (3) through a breakthrough of small spatial extent; and
c. wherein the side surfaces of the walls (4, 6) of the conduit (3) are smooth and mirrored, while the wall (5), which is opposite the wall (2) with the double mirror pairs, has a cylindricity to the longitudinal axis of the conduit (3), wherein the cylindricity has a radius approximately twice as large as a distance between the wall (2) and (5).

8. The device according to claim 3, characterized in that,
a. all pairs of mirrors used are metallic mirrors without a cover or protective layer,
b. wherein relatively very small and elongated mirror segments are primarily used, which leads to easy production of such mirror coatings on the conduit (3).

9. The device according to claim 4, characterized in that,
a. all pairs of mirrors used are metallic mirrors without a cover or protective layer,
b. wherein relatively very small and elongated mirror segments are primarily used, which leads to easy production of such mirror coatings on the conduit (3).

10. The device according to claim 2, characterized in that, for use the conduit (3) is filled with air in order to achieve effective disinfection with the aid of one or a few weak UV light sources.

11. The device according to claim 3, characterized in that, for use the conduit (3) is filled with air in order to achieve effective disinfection with the aid of one or a few weak UV light sources.

12. The device according to claim 4, characterized in that, for use the conduit (3) is filled with air in order to achieve effective disinfection with the aid of one or a few weak UV light sources.

13. The device according to claim 5, characterized in that, for use the conduit (3) is filled with air in order to achieve effective disinfection with the aid of one or a few weak UV light sources.
